(19) United States
(12) Patent Application Publication
(10) Pub. No.: US 2015/0320867 A1
(43) Pub. Date: Nov. 12, 2015

(54) TOPICAL FORMULATIONS AND METHODS FOR THE USE THEREOF

(71) Applicant: COTY INC., New York, NY (US)

(72) Inventors: Isabelle Hansenne, Westfield, NJ (US); Chong Tony WANG, Cedar Knolls, NJ (US)

(73) Assignee: COTY INC., New York, NY (US)

(21) Appl. No.: 14/805,042

(22) Filed: Jul. 21, 2015

Related U.S. Application Data
(63) Continuation of application No. PCT/US2014/012470, filed on Jan. 22, 2014.
(60) Provisional application No. 61/755,373, filed on Jan. 22, 2013.
(60) Provisional application No. 61/807,237, filed on Apr. 1, 2013.

(51) Int. Cl. A61K 47/34; A61K 9/70; A61K 8/67; A61Q 19/00; A61K 8/891; A61K 31/07; A61K 8/02

(52) U.S. Cl. CPC A61K 47/34; A61K 31/07; A61K 9/7007; A61K 8/0208; A61Q 19/00; A61Q 19/007; A61K 8/891; A61K 8/671; A61K 2800/30

(57) ABSTRACT

Provided herein, inter alia, are compositions for topical delivery of active agents via the skin with reduced discomfort caused by drying, irritation and/or inflammation of the skin and surrounding tissues.

### CROSS-REFERENCES TO RELATED APPLICATIONS

This application is a continuation of International Application No. PCT/US2014/012470, filed on Jan. 22, 2014, and entitled “TOPICAL FORMULATIONS AND METHODS FOR THE USE THEREOF,” which is a non-provisional of U.S. Provisional Application No. 61/755,373, Filed Jan. 22, 2013 and of U.S. Provisional Application No. 61/807,237, Filed Apr. 1, 2013.

### BACKGROUND OF THE INVENTION

In accordance with the present invention, there are provided compositions for topical delivery of a wide variety of active agents to a subject in need thereof. In one aspect, the invention relates to methods for the delivery of active agents via the skin. In one aspect, the invention relates to methods for the delivery of active agents via the skin with reduced discomfort caused by drying, irritation and/or inflammation of the skin and surrounding tissues.

In accordance with another aspect of the present invention, there are provided methods for making compositions suitable for topical delivery of a wide variety of active agents.

### BRIEF SUMMARY OF THE INVENTION

The present invention relates, inter alia, to compositions for topical delivery of active agents. In one aspect, the invention relates to methods for the delivery of active agents via the skin. In another aspect, the invention relates to methods for the delivery of active agents via the skin with reduced discomfort caused by drying, irritation and/or inflammation of the skin and surrounding tissues.

### DETAILED DESCRIPTION OF THE INVENTION

In accordance with the present invention, herein are provided compositions which include an active agent and an alkyl polysiloxane that is liquid at room temperature. The composition contains substantially no alkyl-perfluoroalkyl ether. In embodiments, the compositions provided herein are substantially clear or translucent (e.g. not cloudy or hazy when viewed with the naked eye).

In accordance with embodiments of the present invention, there are provided methods for treating skin conditions in subject(s) in need thereof. The methods include applying an effective amount of the herein-described compositions, including embodiments thereof to the skin or mucosal regions of the subject.

In accordance with additional embodiments of the present invention, there are provided methods of making composition(s) for the topical delivery of one or more active agents to the skin and/or mucosal regions of a subject. The methods include combining a first solution including an active agent solubilized in a suitable diluent therefor (e.g., an organic solvent) with a second solution including one or more pharmaceutically acceptable additives. A third solution including an alkyl polysiloxane that is liquid at room temperature is added to the resulting combination.

It is to be understood that the present invention is not limited to particular embodiments described, which may, of course, vary. It is also to be understood that the terminology used herein is for the purpose of describing particular embodiments only and is not intended to be limiting.

In certain aspects, the present invention has utility for topical delivery of a wide variety of active agents. In embodiments, the present invention is directed to the delivery of hydrophobic active agents to the skin with reduced side effects related to the nature of the active agent and/or the solvent employed, as well as improved user comfort and compliance.

Broadly speaking, compositions according to the present invention include an active agent combined with a diluent which facilitates transdermal delivery thereof.

An “alkyl,” by itself or as part of another substituent, means, unless otherwise stated, is a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

A “heteroalkyl,” by itself or in combination with another term, is, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. A heteroalkyl is an uncyclized chain. The heteroatom(s) O, N, P, S, B, As, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

A “cycloalkyl” and “heterocycloalkyl,” by themselves or in combination with other terms, are, unless otherwise stated, cyclic versions of “alkyl” and “heteroalkyl,” respectively. Neither cycloalkyls nor heterocycloalkyls are aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein the term “active agent” refers to a molecule suitable for delivery via the skin or mucosal regions of a subject. In embodiments of the present invention, an active agent has pharmaceutical activity and is present for the treatment or prevention of a skin condition. In embodiments of the present invention, active agents may lack pharmaceutical activity, but instead impart other desirable property(ies), such as for example, moisturizing, retaining moisture, maintaining hair or skin in good condition, deodorizing or odor neutralizing, controlling viscosity or foaming, emulsifying, cleansing, or UV protection. In embodiments, active agents are low polarity molecules such as those having a hydrocarbon chain of three or more carbons, but may also include materials of higher polarity.

Exemplary active agents include vitamin A or its derivatives; vitamin C or its derivatives; vitamin E or its derivatives (e.g. tocopherols); hydroxy acids; emollients; humectants; conditioning agents such silicones; aromatic molecules such as benzoyl peroxide and resorcinol; antimicrobials such as azelaic acid, erythromycin, sodium sulfacetamide, tetracycline and derivatives, clindamycin, and the like; anti-neoplastic agents and/or ophthalmic agents including 5-fluorouracil, doxorubicin, imiquimod, sodium [α-(2,6-dichloranilino)phenyl]acetate, and the like; anti-viral agents including ganciclovir, trifluorothymidine and related compounds; anti-inflammatory agents including nonsteroidal anti-inflammatory agents (NSAIDs), including flurbiprofen, ibuprofen, naproxen, indomethacin and related compounds; anti-mitotic drugs including colchicine, taxol and related compounds; drugs that act on actin polymerization including phalloidin, cytochlasin B and related compounds; inhibitors of dihydropyrimidine dehydrogenase (DPD), thymidine phosphorylase (TP) and/or uridine phosphorylase (UP) enzyme inhibitors; ultraviolet light (UV) filters including benzophenone derivatives such as oxybenzone, octocrylene, octyl methoxycinnamate, and avobenzone; radiation proactive agents including methyluracils such as 6-methyluracil and 4-methyluracil; immunomodulating molecules such as tacrolimus, and pimecrolimus; and the like.

In embodiments, the active agent is a vitamin A or vitamin A derivative. A vitamin A derivative is a compound having structural components of Vitamin A thereby imparting a biological activity similar to vitamin A. Examples of vitamin A or its derivatives contemplated for use herein include retinoids such as retinal, retinoic acid, retinoate, retinyl ester, retinol, tretinoin, isotretinoin, adapalene, tazarotene, and the like. The term “retinoids” includes cis and trans derivatives of retinoids (e.g. all-trans-retinoic acid, 13-cis-retinoic acid, 13-trans retinoic acid, and 9-cis-retinoic acid, and derivatives thereof).

As used herein, “retinyl ester” and “retinoate” refers to retinoids having the formula:

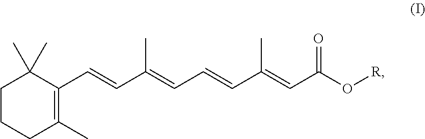

where R is absent (e.g. O⁻), hydrogen (e.g. tretinoin), or substituted or unsubstituted alkyl moiety (e.g. C₁-C₁₀ alkyl). R may be R¹-substituted alkyl, wherein R¹ is independently halogen, oxo (e.g. ═O), —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹ is halogen, oxo, —OH, —NH₂, —CF₃, or substituted or unsubstituted alkyl. Retinyl esters and retinoates useful in the present invention are described in U.S. Pat. No. 4,885,311; U.S. Pat. No. 5,837,728; U.S. Pat. No. 5,124,356; and U.S. Pat. Appl. No. 2008/0139518, which are fully incorporated herein.

R may be a hydroxypinacolone moiety (e.g. MDI 101). R may be a 2-hydroxy-1-(4-methoxyphenyl)ethanone moiety (e.g. MDI 403). In embodiments, the active agent is a retinoate described by formula (I). In embodiments, the active agent is hydroxypinacolone retinoate having formula:

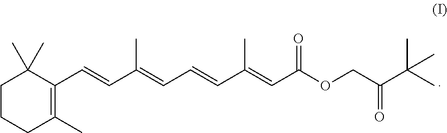 [SMILES: CC1=C(/C=C/C(C)=C/C=C/C(C)=C/C(=O)OCC(=O)C(C)(C)C)C(C)(C)CCC1 ; "."]

In embodiments, the active agent is retinol, retinal, retinoic acid, retinoate, or a retinyl ester. In embodiments, the active agent is a retinoate, e.g. hydroxypinacolone retinoate.

Example of vitamin C or its derivatives contemplated for use herein include ascorbic acid, ascorbate (e.g. Tetrahexyldecyl ascorbate), and the like.

Examples of hydroxy acids contemplated for use herein include beta hydroxy acids such as salicylic acid, acetylsalicylic acid, and the like.

In embodiments, active agent may be vitamin A or a derivative thereof, vitamin C or a derivative thereof, vitamin E or a derivative thereof, an hydroxy acid, benzoyl peroxide, salicylic acid, resorcinol, an antimicrobial, an anti-neoplastic agent, an anti-viral agent, a non-steroidal anti-inflammatory agent, a UV filter, a lipid, or an immunomodulator.

Active agents contemplated for use herein need not have pharmaceutical activity. Therefore, other active agents such as cosmetics, pigments, dyes, and fillers are contemplated for incorporation into invention compositions and delivery by invention methods. It is appreciated that, in embodiments, compositions according to the present invention may include more than one active agent. For example, in embodiments, compositions according to the present invention may contain 2, 3, 4, 5, 6, or more active agents. In embodiments, an active agent may be a pro-drug that is converted in due course to a desired active species in the skin or layer thereof.

In embodiments of the present invention, an active agent may be a lipid such as those suitable for controlling perspiration. Lipids contemplated for use herein typically have an HLB of less than about 12, less than about 8, or less than about 6. Exemplary lipids include glyceryl monostearate, glyceryl monoisostearate, glyceryl monomyristate, glyceryl monoleate, diglyceryl monoisostearate, propylene of glycol monostearate, propylene glycol monoisostearate, propylene glycol monocaprylate, sorbitan monoisostearate, sorbitan monocaprylate, sorbitan monoisostearate, glyceryl monolaurate, glyceryl monocaprylate, glyceryl monocaprate, and the like, as well as mixtures of any two or more thereof. In embodiments, the lipid is glyceryl monolaurate, made available by suppliers like Fitz Chem Corporation under the name MONOMULS 90-L12.

Typically the lipid makes up from about 4 to about 35% of the total weight of the composition. In embodiments, the lipid may include from about 5 to about 20%; and in embodiments, from about 10 to about 15% by weight of the composition, based on total weight of the composition and including all ranges subsumed therein.

Examples of pigments contemplated for use herein include inorganic or organic molecules such as molecules in the form of metal lakes. Pigments are made, for example, of titanium dioxide, zinc oxide, D&C Red No. 36 and D&C Orange No. 17, calcium lakes of D&C Red No. 7, 11, 31 and 34, barium lake of D&C Red No. 12, D&C Red No. 13 strontium lake, aluminum lakes of FD&C Yellow No. 5, of FD&C Yellow No. 6, of D&C Red No. 27, of D&C Red No. 21 and of FD&C Blue No. 1, iron oxides, manganese violet, chromium oxide, ultramarine blue, and the like.

In embodiments, the active agent may be provided in a diluent. In embodiments, a diluent is present from 10 to 75 percent w/v. Diluents contemplated for use herein are alkyl siloxanes that are liquid at room temperature.

The “siloxane” and “polysiloxane” refer to linear (poly)organosilanes having a —Si—O—Si— linkage. Accordingly, an “alkyl siloxane” or “alkyl polysiloxane” may refer a methyl polysiloxane having formula:

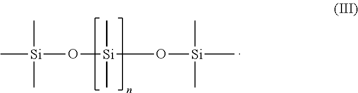

The symbol n is 0, 1, 2, 3, 4, 5, 6, 7, or 8.

Alkyl siloxanes herein are linear and do not include cyclic moieties. Exemplary alkyl siloxanes and alkyl polysiloxanes include hexamethyldisiloxane (e.g. Xiameter® PMX-200 silicon 0.65 cSt, “HMDS”), octamethyltrisiloxane (e.g. Xiameter® PMX-200 silicon 1.0 cSt), decamethyltetrasiloxane (e.g. Xiameter® PMX-200 silicon 1.5 cSt), dodecamethylpentasiloxane (e.g. Xiameter® PMX-200 silicon 2.0 cSt), and ethyltrisiloxane. The term “linear polyorganosilane” refers to a linear branched or unbranched polysiloxane chain as described herein, having 2 to 10 silicon atoms (e.g. a methyl polysiloxane where n is 0 to 8 in Formula (III)). In embodiments, a polyorganosilane refers to polysiloxanes having 2 to 6 silicon atoms (e.g. a methyl polysiloxane where n is 0 to 4 in Formula (III)). In embodiments, a polyorganosilane refers to polysiloxanes having 2 to 4 silicon atoms (e.g. a methyl polysiloxane where n is 0 to 2 in Formula (III)). In embodiments, a polyorganosilane refers to polysiloxanes having 3 to 4 silicon atoms (e.g. a methyl polysiloxane where n is 1 or 2 in Formula (III)). Such compounds are illustratively cyclic silicones or non-cyclic silicones. In embodiments, the diluent is an organic solvent. A “cyclicpolyorganosilane” refers to a polysiloxane wherein at least one silicon atom is substituted with a cyclic moiety (e.g. an unsubstituted 3 to 6 membered cycloalkyl, an unsubstituted 3 to 6 membered heterocycloalkyl, an unsubstituted 5 or 6 membered aryl, or an unsubstituted 5 or 6 membered heteroaryl).

Examples of cyclic silicones include cyclic polydiorganosiloxanes, cyclotetradimethicones, cyclopentadimethicones, and cyclohexadimethicones. Examples of linear organopolysiloxanes include alkyl-, alkoxy- or phenyldimethicones, and alkyl-, alkoxy- or phenyltrimethicones, including embodiments thereof.

Additional diluents contemplated for use herein are volatile aliphatic silicones having from two to six silicon atoms. In one embodiment, an aliphatic volatile silicone is a linear polyorganosiloxane such as a polyorganosiloxane with 2 to 6 silicon atoms, e.g., trisiloxane. In another embodiment, a diluent is ethyl trisiloxane.

In addition, it is appreciated by those of skill in the art that compositions according to the present invention may optionally include more than one diluent, such that the combination of two or more diluents is sufficient to achieve the “liquid at room temperature” property. Combinations contemplated herein include at least a first alkyl polysiloxane. In embodiments, a second alkyl polysiloxane is employed. In embodiments, the first alkyl polysiloxane may be hexamethyldisiloxane, octamethyltrisiloxane, decamethyltetrasiloxane, ethyltrisiloxane (1,1,1,3,5,5,5-heptamethyl-3-ethylsiloxane), or a low viscosity polydimethylsiloxane polymer. The second alkyl polysiloxane may be hexamethyldisiloxane, octamethyltrisiloxane, decamethyltetrasiloxane, ethyltrisiloxane (1,1,1,3,5,5,5-heptamethyl-3-ethylsiloxane), or a low viscosity polydimethylsiloxane polymer. In embodiments, the first and second alkyl polysiloxanes are not the same. In further embodiments, the active agent is vitamin A or derivative(s) (e.g. a retinoate such as MDI 101). In further embodiments, the second alkyl polysiloxane is a polyorganosilane of 2 to 6 silicon atoms. In further embodiments, the second alkyl polysiloxane is a polyorganosilane of 3 or 4 silicon atoms. In further embodiments, the second alkyl polysiloxane is a methyl polysiloxane where n is 1 or 2 in Formula (III). In further embodiments, the second alkyl polysiloxane is octamethyltrisiloxane or decamethyltetrasiloxane. In some embodiments, the first alkyl polysiloxane is not a methyl polysiloxane (e.g. the second alkyl polysiloxane may be ethyl trisiloxane).

The ratio of the first alkyl polysiloxane to the second alkyl polysiloxane may fall in the range of about 1:10 up to about 30:1. The first alkyl polysiloxane may be ethyl trisiloxane. The second alkyl polysiloxane may be decamethyltetrasiloxane (e.g. dimethicone). In embodiments, the first alkyl polysiloxane is ethyl trisiloxane and the second alkyl polysiloxane is decamethyltetrasiloxane. The first alkyl polysiloxane may be present at about 60%, 65%, 70%, 75%, 80%, 85%, 90% or 95% by weight. The first alkyl polysiloxane may be present at about 70% by weight. The first alkyl polysiloxane may be present at about 75% by weight. The first alkyl polysiloxane may be present at about 80% by weight. The first alkyl polysiloxane may be present at about 85% by weight. The first alkyl polysiloxane may be present at about 95% by weight. The first alkyl polysiloxane may be present at about 95% by weight. The first alkyl polysiloxane may be present at about 70% to about 95% by weight. In embodiments, the first alkyl polysiloxane is present at about 72% to about 92% by weight. The first alkyl polysiloxane may be present at about 85% to about 91% by weight. The first alkyl polysiloxane may be present at about 80% to about 92% by weight. The first alkyl polysiloxane may be present at about 88% by weight. In further embodiments, the active agent is vitamin A or derivative(s) (e.g. a retinoate such as MDI 101). In further embodiments, the second alkyl polysiloxane is a polyorganosilane of 2 to 6 silicon atoms. In further embodiments, the second alkyl polysiloxane is a polyorganosilane of 3 or 4 silicon atoms. In further embodiments, the second alkyl polysiloxane is a methyl polysiloxane where n is 1 or 2 in Formula (III). In some embodiments, the first alkyl polysiloxane is not a methyl polysiloxane (e.g. the second alkyl polysiloxane may be ethyl trisiloxane).

In embodiments, the second alkyl polysiloxane is present at about 1%, 2%, 3%, 4%, 5%, 6%, 7%, 8%, 9%, 10%, 11%, 12%, 13%, 14%, 15%, 16%, 17%, 18%, 19%, or 20% by weight. The second alkyl polysiloxane may be present at about 1%, by weight. The second alkyl polysiloxane may be present at about 2%, by weight. The second alkyl polysiloxane may be present at about 3%, by weight. The second alkyl polysiloxane may be present at 4%, by weight. The second alkyl polysiloxane may be present at about 5%, by weight. The second alkyl polysiloxane may be present at about 6%, by weight. The second alkyl polysiloxane may be present at about 7%, by weight. The second alkyl polysiloxane may be present at about 8%, by weight. The second alkyl polysiloxane may be present at about 9%, by weight. The second alkyl polysiloxane may be present at about 10%, by weight. The second alkyl polysiloxane may be present at about 11%, by weight. The second alkyl polysiloxane may be present at about 12%, by weight. The second alkyl polysiloxane may be present at about 13%, by weight. The second alkyl polysiloxane may be present at about 14%, by weight. The second alkyl polysiloxane may be present at about 15%, by weight. The second alkyl polysiloxane may be present at about 16%, by weight. The second alkyl polysiloxane may be present at about 17%, by weight. The second alkyl polysiloxane may be present at about 18%, by weight. The second alkyl polysiloxane may be present at about 19%, by weight. The second alkyl polysiloxane may be present at about 20%, by weight. The second alkyl polysiloxane may be present at about 1% to about 20% by weight. The second alkyl polysiloxane may be present at about 1% to about 10% by weight. The second alkyl polysiloxane may be present at about 2% to about 8% by weight. The second alkyl polysiloxane may be present at about 5% by weight. In further embodiments, the active agent is vitamin A or derivative(s) (e.g. a retinoate such as MDI 101).). In further embodiments, the second alkyl polysiloxane is a polyorganosilane of 2 to 6 silicon atoms. In further embodiments, the second alkyl polysiloxane is a polyorganosilane of 3 or 4 silicon atoms. In further embodiments, the second alkyl polysiloxane is a methyl polysiloxane where n is 1 or 2 in Formula (III). In further embodiments, the second alkyl polysiloxane is octamethyltrisiloxane or decamethyltetrasiloxane and the second alkyl polysiloxane is ethyl trisiloxane. In some embodiments, the second alkyl polysiloxane is not a methyl polysiloxane (e.g. the second alkyl polysiloxane may be ethyl trisiloxane). In some embodiments, the second alkyl polysiloxane is not a methyl polysiloxane (e.g. the second alkyl polysiloxane may be ethyl trisiloxane). In some embodiments, the second alkyl polysiloxane is not a methyl polysiloxane (e.g. the second alkyl polysiloxane may be ethyl trisiloxane).

The second alkyl polysiloxane may be octamethyltrisiloxane. Octamethyltrisiloxane may be present at about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, or 40% by weight. In embodiments octamethyltrisiloxane is present at about 10% to about 30% by weight. Octamethyltrisiloxane may be present at about 20% by weight. In embodiments, the first alkyl polysiloxane is ethyl trisiloxane and the second alkyl polysiloxane is octamethyltrisiloxane. In such instances, the second alkyl polysiloxane may be present at about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40% by weight or preferably at about 20% by weight. In further embodiments, the active agent is vitamin A or derivative(s) (e.g. a retinoate such as MDI 101). In some embodiments, the second alkyl polysiloxane is not a methyl polysiloxane (e.g. the second alkyl polysiloxane may be ethyl trisiloxane).

The second alkyl polysiloxane may be hexamethyldisiloxane. Hexamethyldisiloxane may be present at about 1%, 2%, 3%, 4%, 5%, 6%, 7%, 8%, 9%, 10%, 11%, 12%, 13%, 14%_{,} 15%, 16%, 17%, 18%, 19%, 20%, 25%, 30%, 35%, or 40% by weight. Hexamethyldisiloxane may be present at about 1% by weight. Hexamethyldisiloxane may be present at about 2% by weight. Hexamethyldisiloxane may be present at about 3% by weight. Hexamethyldisiloxane may be present at about 4% by weight. Hexamethyldisiloxane may be present at about 5% by weight. Hexamethyldisiloxane may be present at about 6% by weight. Hexamethyldisiloxane may be present at about 7% by weight. Hexamethyldisiloxane may be present at about 8% by weight. Hexamethyldisiloxane may be present at about 9% by weight. Hexamethyldisiloxane may be present at about 10% by weight. Hexamethyldisiloxane may be present at about 11% by weight. Hexamethyldisiloxane may be present at about 12% by weight. Hexamethyldisiloxane may be present at about 13% by weight. Hexamethyldisiloxane may be present at about 14% by weight. Hexamethyldisiloxane may be present at about 15% by weight. Hexamethyldisiloxane may be present at about 16% by weight. Hexamethyldisiloxane may be present at about 17% by weight. Hexamethyldisiloxane may be present at about 18% by weight. Hexamethyldisiloxane may be present at about 19% by weight. Hexamethyldisiloxane may be present at about 20% by weight. Hexamethyldisiloxane may be present at about 25% by weight. Hexamethyldisiloxane may be present at about 30% by weight. Hexamethyldisiloxane may be present at about 35% by weight. Hexamethyldisiloxane may be present at about 40% by weight. In further embodiments, the active agent is vitamin A or derivative(s) (e.g. a retinoate such as MDI 101). In some embodiments, the first alkyl polysiloxane is not a methyl polysiloxane (e.g. the second alkyl polysiloxane may be ethyl trisiloxane).

In embodiments, hexamethyldisiloxane is present at about 10% to about 40% by weight. In embodiments, hexamethyldisiloxane is present at about 10% to about 30% by weight. Hexamethyldisiloxane may be present at about 10% to about 20% by weight. Hexamethyldisiloxane may be present at about 15% to about 25% by weight. In further embodiments, the active agent is vitamin A or derivative(s) (e.g. a retinoate such as MDI 101).

The first alkyl polysiloxane may be ethyl trisiloxane and the second alkyl polysiloxane may be hexamethyldisiloxane. In such instances, hexamethyldisiloxane may be present by percent weight as described herein. Hexamethyldisiloxane may be present at about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, or 40% by weight or preferably at about 20% by weight. In further embodiments, the active agent is vitamin A or derivative(s) (e.g. a retinoate such as MDI 101).

The second alkyl polysiloxane may be decamethyltetrasiloxane. Decamethyltetrasiloxane may be present at about 1%, 2%, 3%, 4%, 5%, 6%, 7%, 8%, 9%, 10%, 11%, 12%, 13%, 14%, 15%, 16%, 17%, 18%, 19%, or 20% by weight. Decamethyltetrasiloxane may be present at about 1% by weight. Decamethyltetrasiloxane may be present at about 2% by weight. Decamethyltetrasiloxane may be present at about 3% by weight. Decamethyltetrasiloxane may be present at about 4% by weight. Decamethyltetrasiloxane may be present at about 5% by weight. Decamethyltetrasiloxane may be present at about 6% by weight. Decamethyltetrasiloxane may be present at about 7% by weight. Decamethyltetrasiloxane may be present at about 8% by weight. Decamethyltetrasiloxane may be present at about 9% by weight. Decamethyltetrasiloxane may be present at about 10% by weight. Decamethyltetrasiloxane may be present at about 11% by weight. Decamethyltetrasiloxane may be present at about 12% by weight. Decamethyltetrasiloxane may be present at about 13% by weight. Decamethyltetrasiloxane may be present at about 14% by weight. Decamethyltetrasiloxane may be present at about 15% by weight. Decamethyltetrasiloxane may be present at about 16% by weight. Decamethyltetrasiloxane may be present at about 17% by weight. Decamethyltetrasiloxane may be present at about 18% by weight. Decamethyltetrasiloxane may be present at about 19% by weight. Decamethyltetrasiloxane may be present at about 20% by weight. In further embodiments, the active agent is vitamin A or derivative(s) (e.g. a retinoate such as MDI 101).

In embodiments, decamethyltetrasiloxane is present at about 1% to about 20% by weight. In embodiments, decamethyltetrasiloxane is present at about 1% to about 15% by weight. In embodiments, decamethyltetrasiloxane is present at about 1% to about 10% by weight. In embodiments, decamethyltetrasiloxane is present at about 1% to about 9% by weight. In embodiments, decamethyltetrasiloxane is present at about 1% to about 8% by weight. In embodiments, decamethyltetrasiloxane is present at about 1% to about 7% by weight. In embodiments, decamethyltetrasiloxane is present at about 1% to about 6% by weight. In embodiments, decamethyltetrasiloxane is present at about 1% to about 5% by weight. In embodiments, decamethyltetrasiloxane is present at about 2% to about 8% by weight. In embodiments, decamethyltetrasiloxane is present at about 3% to about 9% by weight. In embodiments, decamethyltetrasiloxane is present at about 5% to about 10% by weight. In embodiments, decamethyltetrasiloxane is present at about 5% to about 15% by weight. In embodiments, decamethyltetrasiloxane is present at about 5% to about 20% by weight. In further embodiments, the active agent is vitamin A or derivative(s) (e.g. a retinoate such as MDI 101).

The first alkyl polysiloxane may be ethyl trisiloxane and the second alkyl polysiloxane may be decamethyltetrasiloxane. In such instances, decamethyltetrasiloxane may be present by percent weight as described herein. decamethyltetrasiloxane may be present at about 1%, 2%, 3%, 4%, 5%, 6%, 7%, 8%, 9%, 10%, 11%, 12%, 13%, 14%, 15%, 16%, 17%, 18%, 19%_{, or} 20% by weight or preferably at about 5% by weight. In further embodiments, the active agent is vitamin A or derivative(s) (e.g. a retinoate such as MDI 101).

The second alkyl polysiloxane may include a combination of decamethyltetrasiloxane, octamethyltrisiloxane and hexamethyldisiloxane. Thus, in embodiments, the second alkyl polysiloxane may be decamethyltetrasiloxane with small amounts (e.g. less than about 0.1% each) of octamethyltrisiloxane and hexamethyldisiloxane. In further embodiments, the active agent is vitamin A or derivative(s) (e.g. a retinoate such as MDI 101).

The polysiloxane may be Xiameter® PMX-200 silicon 0.65 cSt (85-100 weight percent hexamethyldisiloxane, HMDS), 1.0 cSt (85-100 weight percent octamethyltrisiloxane), 1.5 cSt (85-100 weight percent decamethyltetrasiloxane), or 2.0 cSt (70-90 weight percent dodecamethylpentasiloxane with <1.0 weight percent dodecamethylcyclohexasiloxane and decamethylcyclopentasiloxane). In embodiments the polysiloxane is Xiameter® PMX-200 silicon 1.5 cSt. In further embodiments, the active agent is vitamin A or derivative(s) (e.g. a retinoate such as MDI 101).

Volatile silicones contemplated for use herein are lightweight diluents that evaporate on application and thus have an elegant, light-weight “feel” on the skin. Volatile silicones are typically limited in their ability to dissolve low polarity (i.e. usually greater than C₇-C₈) organic compounds like retinoids. For example, when relatively low therapeutic levels of retinol (0.1-0.2% w/v) are dissolved in cyclomethicone alone, hazy solutions result due to incomplete solubilization by the silicone fluid.

In accordance with the present invention, it has unexpectedly been discovered that an alkyl-perfluoroalkyl ether component is not necessary to assist in the incorporation of a retinoid into a topical formulation at appropriate therapeutic levels. This discovery is interesting due to the fact that alkyl-perfluoroalkyl ethers are taught in the art as necessary components of formulations used for the topical delivery of active agents such as retinoids.

In view of the observation in the art that silicones are generally poor solvents, one of ordinary skill in the art has no expectation that the use of a specific class of silicones (i.e., alkyl polysiloxanes that are liquid at room temperature), or mixtures thereof, would be successful for effectively solubilizing active agents.

In embodiments, active agent is present in less than 30 percent w/w amounts. In embodiments, active agent is present at a weight percent of 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.5, 0.25, 0.1, 0.05, 0.01, 0.005, 0.001, or 0.0001, as well as any level in between or any range therein. In embodiments, active agent is present at 20 percent w/w. Active agent may be present at 10% w/w. Active agent may be present at 5% w/w. Active agent may be present at 10% w/w. Active agent may be present at 10% w/w. Active agent may be present at 3% w/w. Active agent may be present at 2% w/w. Active agent may be present at 1% w/w. Active agent may be present at 0.5% w/w. Active agent may be present at 0.25% w/w. Active agent may be present at 0.1% w/w. Active agent may be present at 0.05% w/w. Active agent may be present at 0.01% w/w.

Illustratively, when azelaic acid is an active agent it is present at 15 to 25 percent w/w.

In embodiments, vitamin A or derivative(s) (e.g. a retinoate such as MDI 101) thereof may be present at about 0.001 to about 2 percent by weight. In embodiments, vitamin A or derivative(s) thereof may be present at about 0.005 to about 1 percent by weight.

In embodiments, vitamin C or derivative(s) thereof may be present at about 0.5 to about 5 percent by weight.

In embodiments, vitamin E or derivative(s) thereof (e.g. tocopherols) may be present at about 0.05 to about 5 percent by weight.

In embodiments, Imiquimod may be present at 3 to 8 percent by weight.

In embodiments, benzoyl peroxide may be present at 1 to 10 percent by weight.

In embodiments, salicylic acid may be present at 1 to 5 percent by weight.

It is within the skill of the art to determine the optimal level of active agent in either a concentrated solution or a final solution for application.

Numerous skin or systemic conditions are treatable with compositions according to the present invention, including, for example, acne, wrinkles, dryness, eczema, psoriasis, actinic and non-actinic keratoses, rosaceous, among others. For example, U.S. Pat. No. 3,932,665 describes retinal as a therapeutic agent in a method for treating acne by topical application. The disclosure of U.S. Pat. No. 3,932,665 is accordingly hereby incorporated by reference in its entirety. The topical administration of 5-fluorouracil for treatment of keratoses is described in U.S. Pat. No. 4,034,114, the disclosure of which is hereby incorporated by reference in its entirety. The use of compositions according to the present invention reduces the associated side effects that typically accompany topical or ophthalmologic administration of active agents. Other active agents described herein (e.g. vitamin C, vitamin E, azelaic acid, Imiquimod, benzoyl peroxide, and salicylic acid) exhibit well understood properties in treating skin or systemic conditions and would be useful for their identified indications as understood by those skilled in the art.

Compositions according to the present invention are suitable for topical delivery of an active agent. For example, a composition according to the present invention may include a retinol formulated in a suitable diluent, i.e., a diluent such as a volatile silicone. With such a diluent, retinol levels needed to achieve beneficial effects are minimized and the potential for irritant effects to the skin by retinol are greatly diminished. Moreover, retinol is stable when formulated with diluent compositions contemplated herein, in contrast to other conventional carriers.

The compositions of the invention may include 0.005 to 1.0 weight percent retinol, in which case they may be applied directly to the skin, or supplied as more concentrated solution containing higher levels of active agent, in which case prior to application they are diluted by suitable means, e.g., employing a cosmetically acceptable carrier to achieve a desired level such as 0.005 to 1.0 weight percent for retinol. In the formulations of the invention, water may be minimized or eliminated to improve the stability of retinol and to minimize the potential for separation of the oil and water. In embodiments, water is present at less than 2%. One of ordinary skill in the art will recognize that differing levels of active agent will be operable herein depending on the desired final amount of active agent.

In embodiments, compositions formulated as described herein are topically applied to the skin at a concentration which results in application of 0.005 to 1.0 weight percent retinol; or, in embodiments, which results in application of 0.01 to 0.5 weight percent. An active agent may be applied, for example, in the areas where fine lines, wrinkles, dry or inelastic skin or large pores are observed. In embodiments, a moisturizer is applied with or after application of compositions according to the present invention to enhance the tactile comfort associated with application of such compositions and to enhance the wrinkle effacement and other benefits achieved by such compositions. An improved characteristic of compositions according to the present invention is that the use of additional moisturizers is not required.

In embodiments, compositions according to the present invention may be formulated with suitable levels of organic solvent. In embodiments, suitable organic solvent is volatile at ambient temperatures and pressures. In embodiments, less than 35% organic solvent is included; in embodiments, less than 30% organic solvent is included; in embodiments, the level of volatile organic solvent is less than 15 percent w/w. In embodiments, an organic solvent is present at 5% or less w/w. In embodiments, an organic solvent is absent.

Organic solvents contemplated for use herein include alcohols (e.g., ethanol, ethoxydiglycol, isopropyl alcohol, and the like). In embodiments, the organic solvent is ethoxydiglycol, present at 10 percent w/w or less. In embodiments, ethoxydiglycol is present at 3 percent w/w. When present, the level of organic solvent is selected so as to not induce noticeable drying or other toxic effects on the skin. In embodiments, it is appreciated that more than one organic solvent may be present in a composition according to the present invention. It is further appreciated that, in embodiments, the compositions according to the present invention may be entirely ethanol free.

It is a particularly surprising and unexpected discovery of the subject invention that stable solutions of active compounds in the diluent can be prepared with less than 15 percent w/w organic solvent when combined with a diluent at 5 percent to 40 percent w/w. It is particularly surprising that a diluent at 5 percent to 40 percent w/w can promote the formation of a stable soluble solution with less than 5% organic solvent.

In embodiments, compositions according to the present invention may include other additives or pharmaceutical carriers such as, for example, stabilizers such as the anti-oxidant BHT; surfactants such as Laureth-4; anti-oxidants such as vitamins C and E, and Green tea extract (i.e. Camellia sinensis) or SILOX GT from Collaborative Labs, Stony Brook, N.Y.; emollients such as a mixture (or single components) of the emollient sold under the brand name SYMREPAIR available from Symrise, Teterboro, N.J., and the like. One of ordinary skill in the art readily appreciates that additives suitable for use with compositions according to the invention include additives which provide desired flow characteristics, absorption, evaporation, delivery of active agent, conversion of a pro-drug, or other desired characteristic(s).

In embodiments, compositions according to the present invention may also be diluted to the appropriate active agent level for application by using other topically acceptable compounds or vehicles which may be miscible with the retinol or other active agent employed in the practice of the present invention. Other cosmetic additives may be employed, either in the compositions of the invention or in those compositions when diluted with a suitable vehicle.

In embodiments, compositions of the present invention containing retinol achieve moisturizing efficacy, thereby precluding the need for a separate moisturizer. Therefore, in embodiments, compositions of the invention are formulated to include moisturizing components that are compatible with the silicone diluent to a level of up to 35% by weight of the final formulation. Exemplary moisturizing ingredients suitable for use in compositions according to the present invention are illustratively petrolatum, ethylhexyl palmitate, cholesterol fatty acid ceramide, squalene, and the like. The addition of one or more moisturizing components is beneficial under a variety of circumstances, e.g., when compositions according to the present invention are applied to previously dried skin or under conditions where dryness commonly occurs such as in cold climates, or winter months. In embodiments, a moisturizing component is applied where the active agent itself has a drying effect such as when retinol or 5-fluorouracil is applied.

With daily application of a retinol containing composition, skin texture, color and tone will improve. Wrinkles and fine lines will be reduced with minimal irritant effects.

In embodiments, composition according to the invention can be applied to the skin of a subject. A subject may be a patient. A subject may be a mammal such as a human, a non-human primate, a horse, a goat, a cow, a sheep, a pig, a dog, a cat, a rodent, and the like.

Compositions according to the present invention can be provided in a variety of forms, e.g., as a lotion, cream, gel, bar, ointment, or in pad form. In embodiments, the composition is provided in a single use container, the contents of which are applied directly to the stratum corneum of a subject or applied to an applicator pad, which is impregnated therewith, for subsequent delivery to the subject.

In embodiments, a cooling effect is observed upon application of compositions according to the present invention. The phrase “cooling effect” as used herein means reducing the temperature of the skin, typically from about 1 to about 2° C. upon application. The cooling effect includes the effect that results from evaporation of solvent and/or diluent.

Compositions according to the present invention may be administered according to any of a variety of protocols, e.g., one to three times daily. Alternatively, compositions according to the present invention may be delivered once daily. As yet another alternative, compositions according to the present invention may be administered weekly, biweekly, monthly, or any subdivision thereof. It is appreciated that compositions according to the present invention can be administered for an amount of time suitable for efficacy of the active agent. Thus, in embodiments, compositions according to the present invention are administered for one to six weeks. In embodiments, compositions according to the present invention are administered indefinitely.

In another aspect is a method for treating a skin condition in a subject in need thereof. The method includes applying an effective amount of the compositions described herein, including embodiments thereof, to the skin or mucosal regions of the subject. In embodiments, the skin condition treated is acne, wrinkles, dryness, eczema, psoriasis, actinic keratoses, nonactinic keratose, or rosaceous. The effective amount of the compositions may be applied according to the method one time daily, two times daily, three times daily, weekly, biweekly, monthly, or quarterly, or any multiples or combinations thereof. The compositions according to the present invention may be applied according to the method one time daily, two times daily, or three times daily.

Also provided herein is a process for formulating compositions according to the present invention, which achieve pleasing administration to the skin of a subject. Formulation processes according to the present invention contemplate making a first solution by solubilizing one or more active agents optionally in a diluent (e.g. organic solvent). Such solubilizing is preferably performed with gentle mixing in low to no light conditions.

A second solution is made by mixing any desirable optional pharmaceutically acceptable additives such as emollients and vitamins. The second solution is added to and mixed with the first solution. Such mixing is preferably carried out in the dark under gentle mixing conditions.

An optional third solution including an alkyl polysiloxane that is liquid at room temperature is added to the combined first and second solutions to form a composition. The third solution may include two or more polysiloxanes as described herein. The alkyl polysiloxane may be decamethyltetrasiloxane. The decamethyltetrasiloxane may be present at about 5% by weight. Mixing is optionally non-vortex, gentle mixing in low light or darkness. Mixing is preferably for 120 minutes. The composition is preferably stored under inert gas such as nitrogen gas.

It is appreciated that low to no light conditions are important should light sensitive components be present in the subject composition. In the absence of light sensitive components, the process of the present invention is optionally performed in ambient or other lighting conditions.

The formulation process according to the present invention is optionally performed at ambient temperature and pressure conditions. In embodiments, the formulation process according to the present invention is performed by heating one or more components or solutions.

Various aspects of the present invention are illustrated by the following non-limiting examples. The examples are for illustrative purposes and are not a limitation on any practice of the present invention. It will be understood that variations and modifications can be made without departing from the spirit and scope of the invention. One of ordinary skill in the art readily knows how to synthesize or commercially obtain the reagents and components described herein.

The foregoing description is illustrative of particular embodiments of the invention, but is not meant to be a limitation upon the practice thereof. The following claims, including all equivalents thereof, are intended to define the scope of the invention.

### EXAMPLES

### Example 1

### Formulations

### Comparative Formulation I

Comparison Formulation I was prepared by mixed the following components according to the following protocol:

| | |
|---|---|
| 72.3 percent | Silsoft ETS (Ethyl trisiloxane); |
| 20 percent | 3M CF-61 (methoxynonafluorobutane); |
| 1.0 percent | SILOX GT (cyclopentasiloxane, cyclohexasiloxane, |
| | and Camellia Sinensis leaf extract); |
| 2.0 percent | SYMREPAIR 100 (hexyldecanol, bisabolol, |
| | cetylhydroxyproline palmitamide, stearic acid, |
| | and Brassica Campestris sterols); |
| 2.0 percent | BV-OSC (Tetrahexyldecyl ascorbate); |
| 0.50 percent | DL-alpha tocopherol (Tocopherol); |
| 1.5 percent | Transcutol (ethoxydiglycol); |
| 0.5 percent | BRIJ L4-LQ-(AP) (Laureth-4); |
| 0.1 percent | Eutanal G 16 (hexyldecanol); and |
| 0.10 percent | MDI-101 (hydroxypinacolone retinoate). |

CG was purchased from Gattefosse, Toronto, ON, Canada), Laureth-4 (Croda, Edison, N.J.), hydroxypinacolone retinoate (MDI-101, Concert LLC) and BHT by gentle mixing in a propeller mixer using low light conditions.

Solution 2 was prepared separately. Solution 2 includes SYMREPAIR (Symrise, Inc., Teterboro, N.J.) which includes hexyldecanol, bisabolol, cetyl hydroxyproline palmitate, steraic acid, and Brassica campestris sterols. SYMREPAIR was mixed with tetrahexyldecyl ascorbate (BV-OSC, Barnet, Englewood Cliffs, N.J.) and tocopherol USP in a propeller mixer until a clear solution formed.

Solution 1 was combined with solution 2 by slow addition with continuous, non-vortex propeller mixing protecting the solutions from light. Solution 3 was prepared by gentle propeller mixing at ambient temperature.

Solution 3 includes ethyltrisiloxane (Silsoft ETS, Monentiv, Albany, N.Y.), CF-61 (3M Specialty Materials) and SILOX GT (combination of cyclopentasiloxane and Camellia sinesis leaf extract from BASF Beauty Care). The combined solutions 1 and 2 were slowly added to solution 3 the continuous, non-vortex propeller mixing protected from the light. Mixing was continued for 120 minutes.

Formulation I was transferred to opaque holding containers with nitrogen head-space for storage. 60 mL of Formulation I was then transferred to 2 oz. amber glass bottles with a purified nitrogen gas head-space and stored protected from light until used.

### Formulation II

A formulation may be made containing:

| | |
|---|---|
| 69.3 percent | Silsoft ETS (Ethyl trisiloxane); |
| 20 percent | Xiameter PMX-200 Silicon Fluid 0.65 CS |
| | (85-100 weigh percent hexamethyldisiloxane); |
| 1.0 percent | Juvenesence; |
| 1.0 percent | Syleol A; |
| 1.0 percent | Alpha-lopic acid; |
| 1.0 percent | SILOX GT (cyclopentasiloxane, cyclohexasiloxane, |
| | and Camellia Sinensis leaf extract); |
| 2.0 percent | SYMREPAIR 100 (hexyldecanol, bisabolol, |
| | cetylhydroxyproline palmitamide, stearic acid, and |
| | Brassica Campestris sterols); |
| 2.0 percent | BV-OSC (Tetrahexyldecyl ascorbate); |
| 0.50 percent | DL-alpha tocopherol (Tocopherol); |
| 1.5 percent | Transcutol (ethoxydiglycol); |
| 0.5 percent | BRIJ L4-LQ-(AP) (Laureth-4); |
| 0.1 percent | Eutanal G 16 (hexyldecanol); and |
| 0.10 percent | MDI-101 (hydroxypinacolone retinoate). |

Formulation II was prepared essentially as described above in Example 1, except no alkyl-perfluoroalkyl ether is incorporated therein.

### Formulation III

A formulation may be made containing:

| | |
|---|---|
| 69.3 percent | Silsoft ETS (Ethyl trisiloxane); |
| 20 percent | Xiameter PMX-200 Silicon Fluid 1.0 CS |
| | (85-100 weight percent octamethyltrisiloxane); |
| 1.0 percent | Juvenesence; |
| 1.0 percent | Syleol A; |
| 1.0 percent | Alpha-lopic acid; |
| 1.0 percent | SILOX GT (cyclopentasiloxane, cyclohexasiloxane, |
| | and Camellia Sinensis leaf extract); |
| 2.0 percent | SYMREPAIR 100 (hexyldecanol, bisabolol, |
| | cetylhydroxyproline palmitamide, stearic acid, |
| | and Brassica Campestris sterols); |
| 2.0 percent | BV-OSC (Tetrahexyldecyl ascorbate); |
| 0.50 percent | DL-alpha tocopherol (Tocopherol); |
| 1.5 percent | Transcutol (ethoxydiglycol); |
| 0.5 percent | BRIJ L4-LQ-(AP) (Laureth-4); |
| 0.1 percent | Eutanal G 16 (hexyldecanol); and |
| 0.10 percent | MDI-101 (hydroxypinacolone retinoate). |

Formulation III was prepared essentially as described above in Example 1, except no alkyl-perfluoroalkyl ether is incorporated therein.

### Formulation IV

A formulation may be made containing:

| 88.05 percent | Silsoft ETS (Ethyl Trisiloxane); |
|---|---|
| 5.00 percent | Xiameter PMX-200 Silicon Fluid 1.5 CS |
| | (Dimethicone) (85-100% Decamethyltetrasiloxane, |
| | <0.1% octamethyltrisiloxane, <0.1% |
| | hexamethyldisiloxane); |
| 0.50% | Silox GT (Cyclopentasiloxane, Cyclohexasiloxane |
| | and Camellia Sinensis Leaf Extract); |
| 2.00% | SymRepair 100 (hexyldecanol, bisabolol, |
| | cetylhydroxyproline palmitamide, stearic acid, |
| | and Brassica Campestris sterols); |
| 2.00% | BV-OSC (Tetrahexyldecyl ascorbate); |
| 0.50% | DL-alpha Tocopherol (Tocopherol); |
| 1.5% | Transcutol (Ethoxydiglycol); |
| 0.25% | BRIJ L4-LQ-(AP) (Laureth-4); |
| 0.10% | MDI-101 (Hydroxypinacolone retinoate); and |
| 0.10% | Eutanol G 16 (Hexyldecanol) |

Formulation IV was prepared essentially as described above in Example 1, except no alkyl-perfluoroalkyl ether is incorporated therein. Phase 1 (i.e. ethoxydiglycol, laureth-4, hydroxypinacolone retinoate, and hexyldecanol) was slowly added to a separately made Phase 2 (i.e. SymRepair 100, tetrahexyldecyl ascorbate, and DL-alpha tocopherol). The combined Phase 1 and 2 solution were then slowly added to Phase 3 (i.e. ethyl trisiloxane, Xiameter PMX-200 silicon fluid 1.5 CS, and Silox GT).

### Example 2

A sensory/comparison evaluation was performed by using comparison Formulation I or an exemplary composition according to the present invention (e.g. Formulation II) as follows. Ten panelists applied comparison Formulation I or an exemplary composition according to the present invention (e.g. Formulation II) on one half of the face. Each panelist was then asked to rate each of the following attributes on a 5-point intensity scale:
suppleness,
skin texture,
skin texture visibility,
absorption,
ease of application, and
film texture on the skin,

Panelists were then asked to complete a questionnaire (on a 5-point scale) with respect to at least two of the following parameters:
my skin feels moisturized,
my skin feels hydrated, and/or
my skin feels softened.

All panelists in both the Formulation I group and the Formulation II group reported improved moisture in the tested skin areas.

Therefore, the simplified compositions according to the present invention (i.e. containing substantially no alkyl-perfluoroalkyl ether therein) perform as well as the formulations described in the prior art, which require the presence of alkyl-perfluoroalkyl ethers to facilitate solubilization and delivery of the active agent.

### Example 3

The ability of an electric current to flow through the stratum corneum provides an indirect measurement of the corneum's water content. The panelists who participated in the study in Example 2 were assessed for moisturization using a Corneometer (3 readings on each site) on designated randomized, 4 cm² sites on the arms. Application of product involved applying an even film of product to the designated site. Readings were taken 15 minutes, 2, 6 and 24 hours after application. Panelists were asked to refrain from applying any products to the arms, and shower with Ivory soap for five days prior to the start of the study. Panelists were also asked to come to the lab and acclimate, with arms exposed, for 3 minutes prior to each visit, and were not allowed to apply anything to their arms during the study.

For each time point, individual panelist readings (in triplicate) were averaged and a paired t-test is performed comparing the readings, per time point, of the Test site to the Untreated site to determine statistical significance (p≦0.05).

Change from baseline calculations were determined by subtracting the baseline reading from the 1 minute, 2, 6 and 24 hour readings for each panelist. The average of those calculations was reported as the mean change from baseline. A paired t-test was performed comparing the mean change from baseline for all time points of the Test site to the Untreated site to determine statistical significance (p≦0.05).

The objective measurement and substantiation of the stratum corneum's electrical conductivity showed a significant enhancement in facial skin moisture content for both exemplary Formulation II and comparative Formulation I.

### Example 4

A test of the ability of an exemplary formulation (i.e. Formulation II, III or IV), and comparative Formulation I (as described in Example 1) to reduce skin dryness was performed with or without supplemental moisturizer. Twelve panelists who demonstrate skin dryness upon repeated soap washing of the hands were selected to participate in this study. Initially, the panelists induced a condition of dryness by washing their hands with bar soap. The test formulations were applied daily to one hand while the other was left untreated to serve as a control side. Each hand was rated randomly by two trained evaluators who had no knowledge of which hand was treated. The evaluators used a stereomicroscope to assist them with their ratings.

The results of this study demonstrate that the un-moisturized, treated sides showed improved moisture content with both an exemplary formulation (i.e. Formula II, III or IV) and comparative Formulation I. Given the extent of improvement observed, the addition of moisturizer after each application of either comparative Formulation I or an exemplary formulation (i.e. Formulation II) did not appreciably improve the treated skin moisture content. The benefits of either comparative Formulation I or an exemplary formulation (i.e. Formulation II, III or IV) persisted for twenty-four hours after the final treatment indicating that both comparison Formulation I composition and an exemplary formulation (i.e. Formulation II, III or IV) each provided effective long-lasting moisturization.

### Example 5

### Formulation V

A Formulation V solution was prepared wherein the active ingredient is salicylic acid at 2 percent weight percent final. A phase 1 solution was prepared at ambient temperature by combining dimethyl isosorbide at 15% w/w final, ethanol (SD-Alcohol 40-B, 200 proof) at 4.7% w/w final, Laureth-4 at 1% w/w final, and salicylic acid at 2% w/w final. The phase 1 ingredients were combined with continuous non-vortex propeller mixing.

Phase 2 was formed by combination of Methyl perfluorobutyl ether (and) Methyl perfluoroisobutyl ether (CF-61) at 35% w/w final and the remainder ethyl trisiloxane with continuous non-vortex propeller mixing until a clear solution was formed.

Phase 2 was slowly combined with phase 1 with continuous non-vortex propeller mixing. If a hazy solution is observed it clarified upon standing for 24-48 hours at ambient temperature.

Formulation V was stored in 60 ml volumes with absorbent applicator pads.

### Formulation VI

A Formulation VI solution was prepared where the active ingredient was salicylic acid at 2 percent weight percent final. Formulation VI was prepared substantially as described above for the preparation of comparative Formulation V, except a mixture of 69.3% ethyl trisiloxane and 20% hexamethyldisiloxane (in the substantial absence of any alkyl-perfluoroalkyl ether) was employed in the preparation of Phase 2.

### Formulation VII

A Formulation VII solution was prepared wherein the active ingredient was salicylic acid at 2 percent weight percent final. Formulation VII was prepared substantially as described above for the preparation of comparative Formulation I, except a mixture of 69.3% ethyl trisiloxane and 20% octamethyltrisiloxane (in the substantial absence of any alkyl-perfluoroalkyl ether) was employed in the preparation of Phase 2.

### Formulation VIII

A Formulation VIII solution was prepared wherein the active ingredient was salicylic acid at 2 percent weight percent final. Formulation VIII was prepared substantially as described above for the preparation of comparative Formulation IV, except a mixture 88.05% Silsoft ETS (Ethyl Trisiloxane) and 5.00% Xiameter PMX-200 Silicon Fluid 1.5 CS (Dimethicone) in the substantial absence of any alkyl-perfluoroalkyl ether) was employed.

### Example 6

Patients presenting with acne to a dermatologist provided informed consent to a split face test comparing Formulations V (i.e. having an alkyl-perfluoroalkyl ether) and VI (i.e. absence of an alkyl-perfluoroalkyl ether) of Example 5 with a commercially available benzoyl peroxide topical acne treatment of equal active ingredient concentration (STRIDEX POWER PADS, Blistex, Inc. Oak Brook, Ill.).

Fifteen females aged 20 to 39 applied Formulation V, and another fifteen females aged 20 to 39 applied Formulation VI to one side of their faces and the benzolyl peroxide comparator to the other side once daily for two weeks. Each subject was asked to record any side effects such as dryness, irritation, and perceived skin clarification. Each of the Formulation V, Formulation VI, and the comparator demonstrated similar skin clarification. Subjects reported less irritation and improved skin condition on both the Formulation V and Formulation VI treated sides relative to comparator.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

### Example 7

Panelists were assessed to determine whether consumers could determine noticeable differences between Formulation I (i.e. having an alkyl-perfluoroalkyl ether) and Formulation IV (i.e. absence of an alkyl-perfluoroalkyl ether). 100 μL of each Formulations was applied to a pad approximately 5 minutes before the panelists arrived. The panelists were told not to apply foundation or lotion prior to their visit. Each panelist swiped the samples across their cheek and the panelists were told to wait approximately 5 minutes before selecting the sample that was different either through feel or visual appearance.

Surprisingly, approximately 77% of the panelists assessed were unable to distinguish the alkyl-perfluoroalkyl ether Formulation I product from the non-alkyl-perfluoroalkyl ether, Xiameter containing, Formulation IV product. There was no significant or directional statistical differences found. Thus the elimination alkyl-perfluoroalkyl ether and replacement with an alkyl-siloxane had no detrimental effect on a final product and demonstrates that the alkyl-perfluoroalkyl ether is not necessary to assist in the incorporation of a retinoid into a topical formulation at effective levels.

### EMBODIMENTS

### Embodiment 1

A composition comprising: an active agent; and an alkyl polysiloxane that is liquid at room temperature; provided, however, that said composition contains substantially no alkyl-perfluoroalkyl ether.

### Embodiment 2

The composition of embodiment 1, wherein said composition is useful for topical delivery of one or more active agent(s) to the skin or mucosal regions of a subject.

### Embodiment 3

The composition of embodiment 1 or 2, wherein said active agent comprises vitamin A or a derivative thereof, vitamin C or a derivative thereof, vitamin E or a derivative thereof, an hydroxy acid, benzoyl peroxide, salicylic acid, resorcinol, an antimicrobial, an anti-neoplastic agent, an anti-viral agent, a non-steroidal anti-inflammatory agent, a UV filter, a lipid, or an immunomodulator.

### Embodiment 4

The composition of embodiment 1 to 3, wherein said active agent is present at between 0.0001 up to 30 wt %.

### Embodiment 5

The composition of embodiments 1 to 4, wherein said active agent is a retinoid or Vitamin A, or a derivative thereof.

### Embodiment 6

The composition of embodiments 1 to 5, wherein said retinoid, Vitamin A, or derivative thereof is present in the range of 0.001 up to 2 wt %.

### Embodiment 7

The composition of embodiments 1 to 6, wherein said retinoid, Vitamin A, or derivative thereof is present in the range of 0.005 up to 1.0 wt %.

### Embodiment 8

The composition of embodiments 1 to 7, wherein said alkyl polysiloxane that is liquid at room temperature is a linear polyorganosilane having in the range of 2-6 silicon atoms.

### Embodiment 9

The composition of embodiments 1 to 8, wherein said alkyl polysiloxane that is liquid at room temperature comprises hexamethyldisiloxane, octamethyltrisiloxane, decamethyltetrasiloxane, ethyltrisiloxane (1,1,1,3,5,5,5-heptamethyl-3-ethylsiloxane), or a low viscosity polydimethylsiloxane polymer.

### Embodiment 10

The composition of embodiments 1 to 9, wherein said alkyl polysiloxane that is liquid at room temperature comprises a combination of at least a first alkyl polysiloxane and a second alkyl polysiloxane, wherein said first alkyl polysiloxane is selected from the group consisting of hexamethyldisiloxane, octamethyltrisiloxane, decamethyltetrasiloxane, ethyltrisiloxane (1,1,1,3,5,5,5-heptamethyl-3-ethylsiloxane), and a low viscosity polydimethylsiloxane polymer; and said second alkyl polysiloxane is selected from the group consisting of hexamethyldisiloxane, octamethyltrisiloxane, decamethyltetrasiloxane, ethyltrisiloxane (1,1,1,3,5,5,5-heptamethyl-3-ethylsiloxane), and a low viscosity polydimethylsiloxane polymer; wherein the first and second alkyl polysiloxanes are not the same.

### Embodiment 11

The composition of embodiments 1 to 10, wherein the ratio of said first alkyl polysiloxane to said second alkyl polysiloxane falls in the range of about 1:10 up to about 30:1.

### Embodiment 12

The composition of embodiments 1 to 11, wherein said first alkyl polysiloxane is ethyl trisiloxane and said second alkyl polysiloxane is decamethyltetrasiloxane.

### Embodiment 13

The composition of embodiments 1 to 12, wherein said second alkyl polysiloxane is present at about 1% to about 20% by weight.

### Embodiment 14

The composition of embodiments 1 to 13, wherein said second alkyl polysiloxane is present at about 1% to about 10% by weight.

### Embodiment 15

The composition of embodiments 1 to 14, wherein said second alkyl polysiloxane is present at about 2% to about 8% by weight.

### Embodiment 16

The composition of embodiments 1 to 15, wherein said second alkyl polysiloxane is present at about 5% by weight.

### Embodiment 17

The composition of embodiments 1 to 16, wherein said first alkyl polysiloxane is present at about 72% to about 92% by weight.

### Embodiment 18

The composition of embodiments 1 to 17, wherein said first alkyl polysiloxane is present at about 85% to 91% by weight.

### Embodiment 19

The composition of embodiments 1 to 18, wherein said first alkyl polysiloxane is present at about 88% by weight.

### Embodiment 20

The composition of embodiments 1 to 19, wherein said first alkyl polysiloxane is present at about 80% to 92% by weight.

### Embodiment 21

The composition of embodiments 1 to 20, further comprising an organic solvent.

### Embodiment 22

The composition of embodiments 1 to 21, wherein said organic solvent comprises ethanol, isopropyl alcohol, ethoxydiglycol, caprylic triglyceride or capric triglyceride.

### Embodiment 23

The composition of embodiments 1 to 22, wherein said organic solvent is present at less than 5 percent by weight.

### Embodiment 24

The composition of embodiments 1 to 23 in the form of a lotion, cream, gel, bar, ointment or a pad.

### Embodiment 25

The composition of embodiments 1 to 24 in the form of a pad, wherein said pad is impregnated with said composition.

### Embodiment 26

A method for treating a skin condition in a subject in need thereof, said method comprising applying an effective amount of the composition of claim 1 to the skin or mucosal regions of said subject.

### Embodiment 27

The method of embodiment 26, wherein said skin condition comprises acne, wrinkles, dryness, eczema, psoriasis, actinic keratoses, nonactinic or rosaceous.

### Embodiment 28

The method of embodiments 26 to 27, wherein said composition is administered one time daily, two times daily, three times daily, weekly, biweekly, monthly, or quarterly, or any multiples or combinations thereof.

### Embodiment 29

A method of making a composition useful for topical delivery of one or more active agent(s) to the skin or mucosal regions of a subject, said method comprising: combining a first solution comprising an active agent solubilized in a suitable diluent with a second solution comprising one or more pharmaceutically acceptable additives, and adding to the resulting combination a third solution comprising an alkyl polysiloxane that is liquid at room temperature.

### Embodiment 30

The method of embodiment 29, wherein said diluent is an organic solvent.

What is claimed is:

1. A composition comprising:
an active agent; and
an alkyl polysiloxane that is liquid at room temperature;
provided, however, that said composition contains substantially no alkyl-perfluoroalkyl ether.

2. The composition of claim 1, wherein said composition is useful for topical delivery of one or more active agent(s) to the skin or mucosal regions of a subject.

3. The composition of claim 1, wherein said active agent comprises vitamin A or a derivative thereof, vitamin C or a derivative thereof, vitamin E or a derivative thereof, an hydroxy acid, benzoyl peroxide, salicylic acid, resorcinol, an antimicrobial, an anti-neoplastic agent, an anti-viral agent, a non-steroidal anti-inflammatory agent, a UV filter, a lipid, or an immunomodulator.

4. The composition of claim 3, wherein said active agent is present at between 0.0001 up to 30 wt %.

5. The composition of claim 3, wherein said active agent is a retinoid or Vitamin A, or a derivative thereof.

6. The composition of claim 5, wherein said retinoid, Vitamin A, or derivative thereof is present in the range of 0.001 up to 2 wt %.

7. The composition of claim 5, wherein said retinoid, Vitamin A, or derivative thereof is present in the range of 0.005 up to 1.0 wt %.

8. The composition of claim 1, wherein said alkyl polysiloxane that is liquid at room temperature is a linear polyorganosilane of 2-6 silicon atoms.

9. The composition of claim 1, wherein said alkyl polysiloxane that is liquid at room temperature comprises hexamethyldisiloxane, octamethyltrisiloxane, decamethyltetrasiloxane, ethyltrisiloxane (1,1,1,3,5,5,5-heptamethyl-3-ethylsiloxane), or a low viscosity polydimethylsiloxane polymer.

10. The composition of claim 1, wherein said alkyl polysiloxane that is liquid at room temperature comprises a combination of at least a first alkyl polysiloxane and a second alkyl polysiloxane, wherein
said first alkyl polysiloxane is selected from the group consisting of hexamethyldisiloxane, octamethyltrisiloxane, decamethyltetrasiloxane, ethyltrisiloxane (1,1,1,3,5,5,5-heptamethyl-3-ethylsiloxane), and a low viscosity polydimethylsiloxane polymer; and
said second alkyl polysiloxane is selected from the group consisting of hexamethyldisiloxane, octamethyltrisiloxane, decamethyltetrasiloxane, ethyltrisiloxane (1,1,1,3,5,5,5-heptamethyl-3-ethylsiloxane), and a low viscosity polydimethylsiloxane polymer;
wherein the first and second alkyl polysiloxanes are not the same.

11. The composition of claim 10, wherein the ratio of said first alkyl polysiloxane to said second alkyl polysiloxane falls in the range of about 1:10 up to about 30:1.

12. The composition of claim 10, wherein said first alkyl polysiloxane is ethyl trisiloxane and said second alkyl polysiloxane is decamethyltetrasiloxane.

13. The composition of claim 12, wherein said second alkyl polysiloxane is present at about 1% to about 20% by weight.

14. The composition of claim 13, wherein said second alkyl polysiloxane is present at about 1% to about 10% by weight.

15. The composition of claim 14, wherein said second alkyl polysiloxane is present at about 2% to about 8% by weight.

16. The composition of claim 15, wherein said second alkyl polysiloxane is present at about 5% by weight.

17. The composition of claim 12, wherein said first alkyl polysiloxane is present at about 72% to about 92% by weight.

18. The composition of claim 17, wherein said first alkyl polysiloxane is present at about 85% to 91% by weight.

19. The composition of claim 18, wherein said first alkyl polysiloxane is present at about 88% by weight.

20. The composition of claim 19, wherein said first alkyl polysiloxane is present at about 80% to 92% by weight.

21. The composition of claim 1, further comprising an organic solvent.

22. The composition of claim 21, wherein said organic solvent comprises ethanol, isopropyl alcohol, ethoxydiglycol, caprylic triglyceride or capric triglyceride.

23. The composition of claim 21, wherein said organic solvent is present at less than 5 percent by weight.

24. The composition of claim 1 in the form of a lotion, cream, gel, bar, ointment or a pad.

25. The composition of claim 1 in the form of a pad, wherein said pad is impregnated with said composition.

26. A method for treating a skin condition in a subject in need thereof, said method comprising applying an effective amount of the composition of claim 1 to the skin or mucosal regions of said subject.

27. The method of claim 26, wherein said skin condition comprises acne, wrinkles, dryness, eczema, psoriasis, actinic keratoses, nonactinic or rosaceous.

28. The method of claim 26, wherein said composition is administered one time daily, two times daily, three times daily, weekly, biweekly, monthly, or quarterly, or any multiples or combinations thereof.

29. A method of making a composition useful for topical delivery of one or more active agent(s) to the skin or mucosal regions of a subject, said method comprising:
combining a first solution comprising an active agent solubilized in a suitable diluent with a second solution comprising one or more pharmaceutically acceptable additives, and
adding to the resulting combination a third solution comprising an alkyl polysiloxane that is liquid at room temperature.

30. The method of claim 29, wherein said diluent is an organic solvent.
